(19) United States
(12) Patent Application Publication
(10) Pub. No.: US 2008/0312439 A1
(43) Pub. Date: Dec. 18, 2008

(54) Work-Up in the Preparation of High-Purity Triethylenediamine

(75) Inventors: Ortmund Lang, Quirnbach (DE); Jurgen Ciprian, Ludwigshafen (DE); Matthias Rauls, Ludwigshafen (DE); Matthias Frauenkron, Kalmhout (NL); Gunther van Cauwenberge, Temse (BE); Jan Nouwen, Brecht (BE)

Correspondence Address: CONNOLLY BOVE LODGE & HUTZ LLP, 1875 EYE STREET, N.W., SUITE 1100, WASHINGTON, DC, 20006, US

(73) Assignee: BASF Aktiengesellschaft, Ludwigshafen (DE)

(21) Appl. No.: 12/095,868

(22) PCT Filed: Dec. 7, 2006
(86) PCT No.: PCT/EP2006/069445

§ 371 Date: Jun. 2, 2008

(30) Foreign Application Priority Data
Dec. 23, 2005 (DE) 10 2005 061 956.8

(51) Int. Cl. C07D 487/02

(52) U.S. Cl. 544/352

(57) ABSTRACT

The present invention relates to a process for working up triethylenediamine (TEDA) in which TEDA is vaporized and the gaseous TEDA is introduced into a solvent, subsequently crystallized and separated off from this and the mother liquor formed is extracted with an extractant, wherein the raffinate phase obtained after the extraction stage is worked up further by adsorption in which extractant and/or by-products and intermediates present in the raffinate phase are removed therefrom.

The present invention relates to a process for working up a raffinate phase which is obtained in the preparation and work-up of triethylenediamine (hereinafter referred to as TEDA for short).

TEDA is an important catalyst for the production of polyurethane foams. Various processes for its preparation and work-up are known, but these are comparatively costly if the requirements in respect of quality, in particular color and color stability, odor and purity of the TEDA are to be met.

The known processes for preparing TEDA lead to formation of product mixtures which comprise TEDA together with water, by-products such as piperazine and high molecular weight compounds and also possibly solvents used in the reaction. TEDA is usually separated off from these product mixtures by batchwise or continuous fractional distillation or rectification and is usually purified further by crystallization and or recrystallization in a subsequent step.

The handling of crystalline TEDA is made difficult by its hygroscopic and toxic properties. Since TEDA solidifies easily, blockages can occur in the apparatuses and pipes used. However, the formation of TEDA/water hydrates with water which may be present also leads to deposits in the apparatuses used.

The patent applications DE 199 33 850.7 and DE 199 62 455.0 of the applicant describe a process for the preparation and work-up of pure TEDA, in which, inter alia, TEDA is vaporized, the gaseous TEDA is introduced into a solvent and is crystallized from the resulting solution.

The patent application DE 101 00 943.7 describes a process for preparing pure TEDA, in which TEDA is vaporized from a product mixture comprising a solvent or a diluent which has a boiling point at atmospheric pressure in the range from 175 to 250° C. and the gaseous TEDA is introduced into a solvent and obtained as pure TEDA of high quality by subsequent crystallization. After liquid-solid separation, this is obtained as high-purity, crystalline TEDA. The mother liquor obtained comprising TEDA together with undesirable by-products and decomposition products, so that the mother liquor cannot be recirculated directly to the process in order to recover the TEDA present in the mother liquor. In an extraction stage described, the mother liquor is worked up further using an extractant which is immiscible or only slightly miscible with the solvent of the mother liquor and in which TEDA dissolves readily. Predominantly TEDA and undesirable by-products and decomposition products have become concentrated in the extraction phase obtained. The raffinate phase obtained, viz. a mother liquor which has been purified to a certain extent, comprises predominantly solvent and possibly further constituents which prevents the desired recirculation to the process.

A disadvantage of this process has been found to be that the extractant used goes over into the raffinate phase in an amount corresponding to the equilibrium. The raffinate phase thus comprises solvent, traces of TEDA and certain amounts of extractant, as a result of which recirculation into the process leads to problems. In particular, the impurities present can reduce the quality of the TEDA obtained. Formation of sparingly soluble, undesired by-products which lead to formation of deposits in the apparatuses and pipes of the process and consequently lead to blockages and a reduction in the operating time can likewise occur. Particularly when using the preferred extractant water, formation of sparingly soluble TEDA/water hydrates can occur. To reduce these disadvantages, it is therefore customary to discharge part of the raffinate phase from the process and discard it, with the amount discharged depending on the extractant used.

It is an object of the present invention to provide an economical process for working up a raffinate phase obtained in the preparation and work-up of pure TEDA, which allows a solvent used in the process to be virtually completely recirculated so that the consumption of fresh solvent is reduced and at the same time the operating time is increased.

The achievement of this object starts out from a process for working up TEDA in which TEDA is vaporized and the gaseous TEDA is introduced into a solvent, subsequently crystallized and separated off from this and the mother liquor formed is extracted with an extractant. In this process of the invention, the raffinate phase obtained after the extraction is worked up further by adsorption. The raffinate phase which has been worked up in this way can optionally be recirculated to the process without the abovementioned disadvantages. In a preferred embodiment, the extraction/adsorption is carried out so that complete recirculation is possible.

The first step of the process of the invention comprises the process steps of the work-up of TEDA as described in the patent applications DE 199 3 850.7, DE 199 62 455.0, DE 101 00 943.7 and DE-A 101 22 502.4 of the applicant. The processes for working up TEDA described in the patent applications mentioned are an integral part of the process of the invention and are incorporated by reference into the present patent application. The processes are presented again below.

The TEDA to be worked up can be obtained by known processes, for example by reaction of monoethanolamine, diethanolamine, triethanolamine, ethylenediamine, diethylenetriamine, triethylenetetramine, piperazine, N-(2-hydroxyethyl)piperazine, N,N′-bis(2-hydroxethyl)piperazine, N-(2-aminoethyl)piperazine, N,N′-bis(2-aminoethyl)piperazine, morpholine or mixtures thereof over a catalyst, for example metal pyrophosphates, metal phosphates, for example alkaline earth metal monohydrogen-phosphate, zeolites, zirconium phosphates, Al₂O₃, SiO₂, phosphorus-comprising TiO₂ or ZrO₂ at elevated temperature, in general from 250 to 450° C. The pressure is usually from 0.1 to 50 bar, in particular from 0.1 to 5 bar. The reaction can optionally be carried out in the presence of an inert polar aprotic solvent such as N-alkylpyrrolidones, for example N-methylpyrrolidone, dioxane, THF, dialkylformamides, for example dimethylformamide, dialkylacetamides, for example dimethylacetamide, and an inert carrier gas, for example N₂ or Ar.

The TEDA prepared by a known process is vaporized from the product mixture present and the gaseous TEDA is immediately introduced into a suitable solvent (TEDA quench). The formation of undesirable by-products which lead to a reduction in the quality of the TEDA is significantly reduced in this way.

Many organic solvents are suitable as solvents for the TEDA quench. Examples comprise aliphatic, cyclic or acyclic hydrocarbons, in particular cyclic and acyclic, branched or unbranched alkanes or alkane mixtures, for example n-pentane, i-pentane, cyclopentane, hexane, cyclohexane, heptane, octane and petroleum ether, chlorinated aliphatic hydrocarbons, in particular chlorinated alkanes, for example dichloromethane, trichloromethane, dichloro ether, trichloro ether, aromatic hydrocarbons, for example benzene, toluene and xylenes, chlorinated aromatic hydrocarbons, for example chlorobenzene, alcohols, for example methanol, ethanol, ethylene glycol, 1,4-butanediol and polyether alcohols, in particular polyalkylene glycols, for example diethylene glycol and dipropylene glycol, ketones, for example acetone, methyl ethyl ketone and diethyl ketone, aliphatic carboxylic esters, for example ethyl acetate, aliphatic nitrites, for example acetonitrile and propionitrile, ethers, for example dioxane, THF, diethyl ether and ethylene glycol dimethyl ether and also mixtures of the abovementioned solvents.

Preference is given to using an aliphatic hydrocarbon or a polyalkylene glycol, in particular a saturated cyclic or acyclic, aliphatic hydrocarbon having from 5 to 8 carbon atoms, for example pentane, hexane, cyclohexane or heptane, or dipropylene glycol as solvent for the TEDA quench.

The introduction of the gaseous TEDA into the liquid solvent is carried out in a quenching apparatus, preferably a falling film condenser (thin film condenser, trickle film condenser or falling film condenser) or in a nozzle apparatus. The gaseous TEDA can be conveyed in cocurrent with or countercurrent to the liquid solvent. It is advantageous to introduce the gaseous TEDA into the quenching apparatus from the top. Furthermore, it is advantageous to feed the solvent in tangentially at the top of the falling film condenser or to feed the liquid solvent in through one or more nozzles in order to achieve complete wetting of the interior wall of the quenching apparatus.

The amount of solvent used is selected according to what is advantageous in the particular situation. In general, the solvent is employed in such an amount that, depending on the type of solvent, solutions having a TEDA content of from about 1 to 50% by weight, preferably from 20 to 40% by weight, are obtained.

In general, the temperature in the TEDA quench is set to from 20 to 100° C., preferably from 30 to 60° C., by heating the solvent used and/or the quenching apparatus.

The absolute pressure in the TEDA quench is generally from 0.5 to 1.5 bar.

The TEDA is subsequently crystallized from the solution obtained. The crystallization of the TEDA can be effected by one of the methods known to those skilled in the art. The TEDA crystals obtained by means of a subsequent multistage, preferably single-stage, crystallization are highly pure and are isolated by solid-liquid separation.

The mother liquor present after this is brought into intimate contact with an extractant in an extraction stage in order to recover TEDA still present and also to separate off any impurities comprised.

The extractant is selected so that the TEDA and the by-products and decomposition products responsible for reducing the quality of the TEDA go over virtually completely into the extract phase. In this way, the mother liquor is freed of these by-products and decomposition products, which is a prerequisite for recirculation into the process.

The solvent used as extractant advantageously displays the following properties:

The extractant forms a large miscibility gap with the solvent used for the TEDA quench, with the mutual solubility of extractant and solvent being <1.0% by weight, preferably <1% by weight. TEDA dissolves significantly better in the extractant than in the solvent used in the crystallization stage.

The extractant and the mother liquor have a sufficient density difference, which aids separation of extract phase and raffinate phase. The density difference is preferably >50 kg/m³, in particular >100 kg/m³.

The solvents which are preferably used as extractants according to the invention have hydroxyl groups and are in particular water or water-miscible solvents such as lower alcohols or dihydric or polyhydric alcohols. Examples of suitable alcohols comprise methanol, ethanol, n-propanol, i-propanol, ethylene glycol, polyethylene glycol and glycerol. The alcohols can be used individually or as a mixture, if appropriate also in admixture with water. The most preferred extractant is water.

The mentioned decomposition products could be further referred to as decomposition products as well as intermediates.

After the mass transfer of the extraction has occurred, the mother liquor which has been depleted in TEDA, by-products and decomposition products (raffinate phase) and the extractant enriched with TEDA and the by-products and intermediates (extract phase) are separated in an appropriate manner.

Since not only the TEDA but also the by-products and decomposition products formed in its preparation and work-up go over into the extract phase in the extraction, a raffinate phase which is mother liquor which is now largely free of TEDA and the undesirable by-products and intermediates is obtained in this way. It is desirable for the mother liquor to be recirculated virtually completely to the process and be reused as solvent for the TEDA quench.

Owing to the equilibrium established in the extraction, the raffinate phase comprises proportions of the extractant, as a result of which direct recirculation of this raffinate phase to the process is not possible. The impurities present can reduce the quality of the TEDA and formation of undesirable by-products which cause a reduction in the operating time of the work-up process can occur.

The process of the invention separates the contaminating components, in particular the dissolved extractant, off from the raffinate phase. This work-up step can be configured as a distillation, a membrane process or an adsorption. According to the invention, preference is given to an adsorption which can easily be integrated into the overall process sequence for the preparation and work-up of TEDA in the form of a continuous or batchwise adsorption stage.

The adsorption according to the invention reduces the amount of extractant comprised in the raffinate phase to values of <1 ppm by weight, preferably <0.1 ppm by weight, particularly preferably <0.01 ppm by weight. This is achieved according to the invention by contact of the raffinate phase with one or more suitable adsorbents. Suitable adsorbents are those which are suitable for use in the liquid phase.

Furthermore, preferred adsorbents are those which result in the extractant which is present in only small amounts in the raffinate phase being removed virtually completely or down to a tolerable content of <1 ppm by weight. In particular, the molecule groups comprised in the preferred system, i.e. saturated hydrocarbon as solvent and an extractant having one or more hydroxyl groups, particularly preferably pentane and water, have comparable molecular sizes. The adsorbent should accordingly have, in particular, hydrophilic properties in order to adsorb the extractant which preferably has hydroxyl groups. Suitable adsorbents under these circumstances are silica gel or molecular sieves. Preference is given to using a molecular sieve, preferably a crystalline zeolite, having a suitable pore radii distribution and advantageous hydrophilic properties. Particular preference is given to using a molecular sieve having a pore radius of 3 Angstrom and a specific surface area of 1000 m²/g. The adsorbent used according to the invention should particularly preferably remove the extractant water from the solvent pentane down to a content of 0.01 ppm by weight, with the raffinate stream being fed to the adsorption at a velocity of from 1 to 10 m/h.

The temperature at which the treatment according to the invention of the raffinate phase with the adsorbents is carried out is from 10 to 100° C., preferably from 20 to 40° C., and the pressures are from 0.1 to 5 bar, preferably from 0.5 to 1.5 bar.

In a preferred embodiment of the present invention, the adsorbents are present in the form of a fixed bed and the raffinate phase to be worked up is passed over the fixed bed. The absorbents can also be used in the form of a suspended bed.

The process can be carried out continuously, semicontinuously or batchwise.

When a particular degree of saturation of the adsorbent is reached, this laden adsorbent can be replaced by unladen adsorbent. In a further embodiment, the adsorbents can be regenerated by removing the adsorbed components at elevated temperature and/or by stripping with an inert gas. The preferred process variant is chosen from an economic point of view. The molecular sieve used according to the invention, in particular, has a long active phase as a result of which regeneration steps are necessary only after a long period of operation.

The raffinate phase which has been worked up by the process of the invention can be recirculated virtually completely to the process stage of the TEDA quench and thus reduces the amount of fresh solvent required. In addition, the formation of undesirable by-products, in particular the sparingly soluble TEDA/water hydrates which are predominantly formed when water is used as extractant and lead to a reduction in the operating time of the work-up plant is avoided.

As a result of the work-up of the raffinate phase and the sought-after complete recirculation of the solvent, the TEDA quality and also the operating time of the apparatuses and the pipes in the work-up are increased compared to the previous ways of carrying out the process.

In a preferred embodiment, the process of the invention can, for example, be carried out as follows:

A mixture comprising TEDA, which is obtained, for example, as reaction discharge in a continuous process from the reaction of ethylenediamine and piperazine in a gas-phase reactor at from 320 to 420° C. and from 0.5 to 1.5 bar in the presence of a solvent (for example water), a carrier gas (for example N2 or Ar) and a zeolite catalyst, is fed into a distillation column having about 15 theoretical plates in a distillation stage. Here, low boilers (for example ammonia, ethylamine, water) are separated off overhead at a temperature at the top of from 95 to 120° C. and a pressure of generally from 500 mbar to 1.5 bar. The bottoms are pumped into a further distillation column having about 30 theoretical plates. At a temperature at the top of from 140 to 160° C. and a pressure of from 500 mbar to 1.5 bar, piperazine is separated off overhead in this column and optionally recirculated to the synthesis reactor.

The bottoms comprising TEDA and high boilers are pumped into a further distillation column having about 25 theoretical plates. At a pressure of generally from 500 mbar to 1.5 bar, the high boilers are discharged via the bottoms in this column. At the top of the column, TEDA having a purity of >95% by weight, in particular >97% by weight, is taken off in gaseous form via a partial condenser and is directly shock-cooled and simultaneously dissolved in a solvent, preferably pentane and/or cyclohexane, at a temperature of from 30 to 100° C., preferably from 30 to 60° C., in a falling film condenser (TEDA quench).

After the TEDA quench, TEDA is crystallized out from the resulting solution in a crystallization stage by evaporation of the solvent at a temperature of from 10 to 100° C., preferably from 20 to 40° C., and a pressure of from 0.1 to 5 bar, preferably from 0.5 to 1.5 bar, or by cooling at a temperature of generally from −10 to 40° C., preferably from 0 to 10° C.

The suspension taken off from the crystallization stage is separated into high-purity, crystalline TEDA and mother liquor in a solid-liquid separation, for example in a centrifuge. The mother liquor, which still comprises residues of the desired product and also by-products and intermediates, is then brought into intimate contact with the extractant, preferably water, at a temperature of from 10 to 100° C., preferably from 20 to 40° C., and a pressure of from 0.1 to 5 bar, preferably from 0.5 to 1.5 bar, in an extraction stage, for example a mixer/settler or an extraction column.

The extract phase leaving the extraction stage after mass transfer and subsequent phase separation, which comprises the major part of the TEDA and the undesirable by-products and decomposition products which can lead to a reduction in the quality of TEDA, is recirculated either directly back to the reactor or via a distillation stage to the purification. The raffinate phase, which comprises the solvent together with the dissolved extractant and now only traces of TEDA and by-products and decomposition products, is passed over an adsorbent, preferably a 3 Å molecular sieve, at a temperature of from 10 to 100° C., preferably from 20 to 40° C., and a pressure of from 0.1 to 5 bar, preferably from 0.5 to 1.5 bar, in an adsorption stage. The solution obtained from the adsorption stage after mass transfer, which comprises up to an amount of preferably <1 ppm by weight of extractant, is recirculated to the TEDA quench.

The invention is illustrated by the following examples.

### EXAMPLES

### Example 1

### Comparative Example

The cyclohexane solution of TEDA and secondary components, for example ethylpiperazine, piperazine and aminoethylpiperazine, which is obtained from a crystallization stage is extracted continuously with water in a mixer/settler apparatus. The residence time is 10 minutes, the temperature is 20° C. and the pressure is atmospheric pressure. The extract phase comprises water, TEDA and the major part of the secondary components. The raffinate phase obtained has the following composition (figures in % by weight):

| | |
|---|---|
| Cyclohexane | 99.69% |
| Piperazine | 0.01% |
| Aminoethylpiperazine | 0.06% |
| Triethylenediamine | 0.10% |
| Water | 0.14%. |

The high proportion of water in the raffinate phase prevents direct recirculation of the raffinate phase to the process.

### Example 2

### According to the Invention

The experiment is carried out as described in example 1, except that the raffinate phase is passed from the bottom through a vertical adsorption column which has a diameter of 30 mm and a length of 1 m and is filled with a molecular sieve having a pore radius of 3 Å and then analyzed. The adsorption is carried out at a temperature of 20° C. and atmospheric pressure. The superficial velocity of the raffinate phase in the adsorption column is 5 m/h. The solution obtained after the adsorption has the following composition:

| | |
|---|---|
| Piperazine | <1 ppm by weight |
| Aminoethylpiperazine | <1 ppm by weight |
| Triethylenediamine | <1 ppm by weight |
| Water | <1 ppm by weight |
| Balance | cyclohexane. |

### Example 3

### Comparative Example

The pentane solution comprising TEDA and secondary components, for example ethylpiperazine, piperazine and aminoethylpiperazine, which is obtained from a crystallization stage is extracted with water. The raffinate phase obtained has the following composition (figures in % by weight):

| | |
|---|---|
| Pentane | 99.89% |
| Piperazine | 0.01% |
| Aminoethylpiperazine | 0.03% |
| Triethylenediamine | 0.05% |
| Water | 0.02%. |

Recirculation of this solution to the TEDA quench leads to formation of sparingly soluble TEDA hexahydrates which leads to blockage of pipes.

### Example 4

### According to the Invention

The experiment is carried out as described in example 3, except that the raffinate phase is passed from the bottom through a vertical adsorption column which has a diameter of 30 mm and a length of 1 m and is filled with a molecular sieve having a pore radius of 3 Å and then analyzed. The adsorption is carried out at a temperature of 20° C. and atmospheric pressure. The superficial velocity of the raffinate phase in the adsorption column is 5 m/h. The solution obtained at the outlet of the adsorption column has the following composition:

| | |
|---|---|
| Piperazine | <1 ppm by weight |
| Aminoethylpiperazine | <1 ppm by weight |
| Triethylenediamine | <1 ppm by weight |
| Water | <1 ppm by weight |
| Balance | pentane. |

Recirculation of this solution to the TEDA quench does not lead to any formation of sparingly soluble TEDA hexahydrates, so that blockage of the pipes is avoided.

1. A process for working up triethylenediamine (TEDA) in which TEDA is vaporized and the gaseous TEDA is introduced into a solvent, subsequently crystallized and separated off from this and the mother liquor formed is extracted with an extractant, wherein the raffinate phase obtained after the extraction stage is worked up further by adsorption.

2. The process according to claim 1, wherein the adsorption removes the extractant present in the raffinate phase and/or by-products and intermediates formed in the preparation and work-up of TEDA which are present.

3. The process according to claim 1, wherein the raffinate phase after the adsorption comprises a proportion of <1 ppm by weight of extractant and/or by-products and intermediates.

4. The process according to claim 1, wherein the raffinate phase after the adsorption comprises a proportion of <0.1 ppm by weight of extractant and/or by-products and intermediates.

5. The process according to claim 1, wherein the raffinate phase comprises a solvent in the form of a saturated hydrocarbon, an extractant having hydroxyl groups and/or by-products and intermediates.

6. The process according to claim 1, wherein a molecular sieve is used as adsorbent in the absorption.

7. The process according to claim 6, wherein the molecular sieve has a pore radius of 3 Angstrom.

8. The process according to claim 1, wherein the raffinate phase which has been purified by adsorption is recirculated to the process for the work-up of TEDA.

9. The process according to claim 1, wherein the absorbent is present in the form of a fixed bed, a fluidized bed or a suspended bed.

10. The process according to claim 1, which is carried out continuously, semicontinuously or batchwise.

11. The process according to claim 2, wherein the raffinate phase after the adsorption comprises a proportion of <1 ppm by weight of extractant and/or by-products and intermediates.

12. The process according to claim 2, wherein the raffinate phase after the adsorption comprises a proportion of <0.1 ppm by weight of extractant and/or by-products and intermediates.

13. The process according to claim 3, wherein the raffinate phase after the adsorption comprises a proportion of <0.1 ppm by weight of extractant and/or by-products and intermediates.

14. The process according to claim 2, wherein the raffinate phase comprises a solvent in the form of a saturated hydrocarbon, an extractant having hydroxyl groups and/or by-products and intermediates.

15. The process according to claim 3, wherein the raffinate phase comprises a solvent in the form of a saturated hydrocarbon, an extractant having hydroxyl groups and/or by-products and intermediates.

16. The process according to claim 4, wherein the raffinate phase comprises a solvent in the form of a saturated hydrocarbon, an extractant having hydroxyl groups and/or by-products and intermediates.

17. The process according to claim 2, wherein a molecular sieve is used as adsorbent in the absorption.

18. The process according to claim 3, wherein a molecular sieve is used as adsorbent in the absorption.

19. The process according to claim 4, wherein a molecular sieve is used as adsorbent in the absorption.

20. The process according to claim 5, wherein a molecular sieve is used as adsorbent in the absorption.
